(19) United States
(12) Patent Application Publication
(10) Pub. No.: US 2016/0015446 A1
(43) Pub. Date: Jan. 21, 2016

(54) ELECTROSURGICAL HANDHELD INSTRUMENT WITH ENHANCED FUNCTIONALITY

(71) Applicant: OLYMPUS WINTER & IBE GMBH, Hamburg (DE)

(72) Inventors: Ilja ASSMUS, Postdam (DE); Tino KIRFE, Berlin (DE)

(21) Appl. No.: 14/768,329

(22) PCT Filed: Feb. 13, 2014
(86) PCT No.: PCT/EP2014/052839

§ 371 Date: Aug. 17, 2015

(30) Foreign Application Priority Data
Feb. 15, 2013 (DE) 10 2013 202 526.2

(51) Int. Cl. A61B 18/12; A61B 19/00

(52) U.S. Cl. CPC A61B 18/1206; A61B 19/44; A61B 2018/00178

(57) ABSTRACT

Electrosurgical instrument including a handpiece which has a connection side (A) and a treatment side (B), and including a connecting plug which is electrically connected to the connection side (A) of the handpiece and is electrically connectable to a high-frequency generator, wherein the electrosurgical instrument includes a control unit which is designed to actively control the voltage waveform of high-frequency energy which is output on the treatment side (B) of the handpiece.

The present invention relates to an electrosurgical handheld instrument comprising a handpiece which has a connection side and a treatment side, and comprising a connecting plug which is electrically connected to the connection side of the handpiece and is electrically connectable to a high-frequency generator which is specified for operating the instrument. US 2011/0071520 discloses an electrosurgical handheld instrument having a transceiver unit integrated into its handpiece which is connected to a microprocessor for bidirectional communication with a high-frequency generator.

A high-frequency generator having a universal connecting socket which comprises an instrument identification is also known from the related art. The instrument identification is used to set parameters for a handheld instrument in the high-frequency generator. For this purpose, the parameters are stored in a non-volatile memory (EEPROM) in the plug of the handheld instrument during manufacture, and are read out and set via the connecting socket when the instrument is connected to the high-frequency generator.

The object of the present invention is to provide an electrosurgical handheld instrument which may be used in a particularly versatile manner.

This object is achieved in that the electrosurgical instrument comprises a control unit which is adapted to actively control the voltage waveform of high-frequency energy which is output on the treatment side of the handpiece.

The present invention has recognized that it is disadvantageous that newly developed electrosurgical handheld instruments may have a broader range of functions than existing high-frequency generators and are therefore often not usable to the full extent. This is due to the fact that the integration of new functions into a high-frequency generator, which would be necessary for operating a newly developed electrosurgical handheld instrument, typically requires a costly hardware modification or new development of the high-frequency generator; therefore, the development of high-frequency generators lags behind that of handheld instruments.

The present invention also includes the recognition that handheld instruments and applicators are “passive” instruments for high-frequency generators. Thus, for example, on bipolar handheld instruments, corresponding connecting plugs having two connectors are provided, and the voltage is routed to the electrodes in two cables. All functions, for example, the waveform, are dependent on the range of functions and the connection options of the high-frequency generator. Active signal adjustment or switching does not take place in the handheld instrument, but is output functionally by the high-frequency generator. The functions are therefore present in the particular generator socket.

Although known high-frequency generators have instrument identification, it is used only for reading out/setting parameters. Active communication between the high-frequency generator and the handheld instrument in order to provide further functions does not occur.

The present invention provides the basis for being able to expand a high-frequency surgery system (handheld instrument and high-frequency generator) to new high-frequency applications in the future, additionally or exclusively in terms of hardware in the handheld instrument, apart from a comparatively economical software update in the high-frequency generator. A costly hardware modification or new development of high-frequency generators is not mandatory. It is also conceivable that the development of special small-scale production instruments or devices having additional functions (for example, motors, valves, pumps, etc.) may also be carried out in a more economical manner, since the functions do not have to be implemented in terms of hardware in the high-frequency generator.

In one preferred refinement, the control unit is adapted to modify high-frequency energy supplied by the high-frequency generator as required and to output this modified high-frequency energy on the treatment side of the handpiece. Advantageously, such modified high-frequency energy may be output on the treatment side of the handpiece without necessarily having to access a function in the high-frequency generator. Modification may also be understood to mean in particular a modulation of high-frequency energy, for example, the impression of a voltage waveform. In the present case, a voltage waveform is to be understood to mean both the amplitude and the frequency of high-frequency energy. If, for example, the high-frequency generator is adapted to provide a first number of voltage waveforms to the instrument, for example, a voltage waveform for cutting and a voltage waveform for coagulating, the electrosurgical instrument is now able via the active control device, for example, to add a third voltage waveform which is not provided by the generator, for example, a voltage waveform for a plasma blend mode.

In one particularly preferred refinement, the instrument has a feedback channel to the high-frequency generator, and the control unit is adapted to control a voltage waveform of the high-frequency energy which is output by the high-frequency generator to the instrument via the feedback channel This means that a signal adjustment, i.e., the modification and/or modulation of high-frequency energy into a desired voltage waveform, does not necessarily have to take place within the instrument itself. Rather, in the present refinement, the control unit situated in the instrument acts as an “external control unit” for a high-frequency generator.

In order to be able to operate the instrument on a wide range of different high-frequency generators, it has proven to be advantageous if the control unit is adapted to switch between different high-frequency voltage modes which are output by the high-frequency generator to the instrument. Advantageously, it is thus possible to access predefined high-frequency voltage modes or voltage waveforms in the high-frequency generator. For example, it is possible to switch between a cutting mode and a coagulation mode. Typically, a hand switch is also provided on an electrosurgical instrument, via which a manual input may take place, in order to switch between different high-frequency voltage modes which are output by the high-frequency generator. The advantage of an active control unit now results from the fact that switching regimes are possible via this control unit which, for example, are not able to be implemented via manual operation of a hand switch. Thus, for example, it is possible to switch between a cutting mode and a coagulation mode having a frequency of 50 Hz.

In one particularly preferred refinement, the feedback channel is provided via a modulated hand switch signal. This has the advantage that a hand switch signal which is typically provided in any case may also be used for transmitting control information from the instrument to the high-frequency generator. A modulation of the hand switch signal may, for example, take place via a modification of the switching frequency between 200 and 300 Hz, for example, via an amplitude or frequency modulation of the switching signal.

In order to ensure safe operation of the electrosurgical instrument, the control unit may be synchronized with a change of the voltage waveform of the high-frequency energy which is output by the high-frequency generator, via a modulated switch signal.

In an additional advantageous refinement, the control unit may be controllable via a modulated hand switch signal which is output by the high-frequency generator. This is recommended, for example, if the high-frequency generator is equipped with enhanced switching functionality via a software update which is comparatively simple to carry out, but the high-frequency generator has no internal driver stage for outputting the enhanced functionality. This driver stage may now also be “transferred” to the control unit in order to output a voltage waveform on the treatment side of the handpiece which the high-frequency generator cannot readily provide.

In order to be able to adapt an instrument in a particularly compact manner, it has proven to be advantageous to supply the control unit with energy via a hand switch line. Alternatively, the control unit may also be supplied with energy via a battery provided in the instrument.

In one particularly preferred refinement, the control unit comprises a microcontroller which is adapted to output an instrument identification of the electrosurgical instrument to a high-frequency generator, and/or the microcontroller is adapted to emulate an instrument identification of a passive non-volatile memory with respect to a high-frequency generator.

As a result, a backward compatibility of the electrosurgical instrument with instruments according to the related art may be ensured. When initializing the handheld instrument, a microcontroller contained by the control unit may, for example, initially emulate the protocol of a passive, nonvolatile memory (EPROM) used in a conventional electrosurgical instrument, and thus to register the instrument to a high-frequency generator.

On the one hand, the control unit may comprise a microcontroller or a similar programmable component. On the other hand, the control unit itself may be adapted as a microcontroller.

Preferably, the control unit has power electronics and/or a driver stage which is/are suitable for impressing a voltage waveform onto high-frequency power.

In order to facilitate the handling of the electrosurgical instrument during an operation, it has proven to be advantageous to situate the control unit in the connecting plug. Alternatively or additionally, the control unit may be situated in the handpiece itself.

In order to enable the connection of the electrosurgical instrument to a plurality of high-frequency generators, the connecting plug may be adapted as a universal plug. In an additional preferred refinement, the control unit is adapted to control treatment electrodes contained by the handpiece. If the electrosurgical instrument is, for example, a bipolar instrument, the control unit is advantageously adapted to operate the treatment electrodes in a bipolar manner using high-frequency energy or a high-frequency voltage waveform.

In an additional preferred refinement, the control unit is adapted to control an electromechanical functional element, preferably a motor, valve, pump, or the like. Since such electromechanical functional elements are typically provided in special small-scale production instruments for which no new development of a high-frequency generator is customary, the aforementioned advantage of potentially omitting a costly hardware modification of the high-frequency generator correspondingly results.

The object is also achieved via a high-frequency surgical device which comprises an electrosurgical instrument and a high-frequency generator described above, wherein the electrosurgical instrument is connectable to the high-frequency generator. The high-frequency surgical device may be refined according to the described refinements with reference to the electrosurgical instrument. The described advantages apply accordingly. The various advantageous refinements may be combined in any arbitrary manner. The present invention is now to be explained in greater detail based on exemplary embodiments.

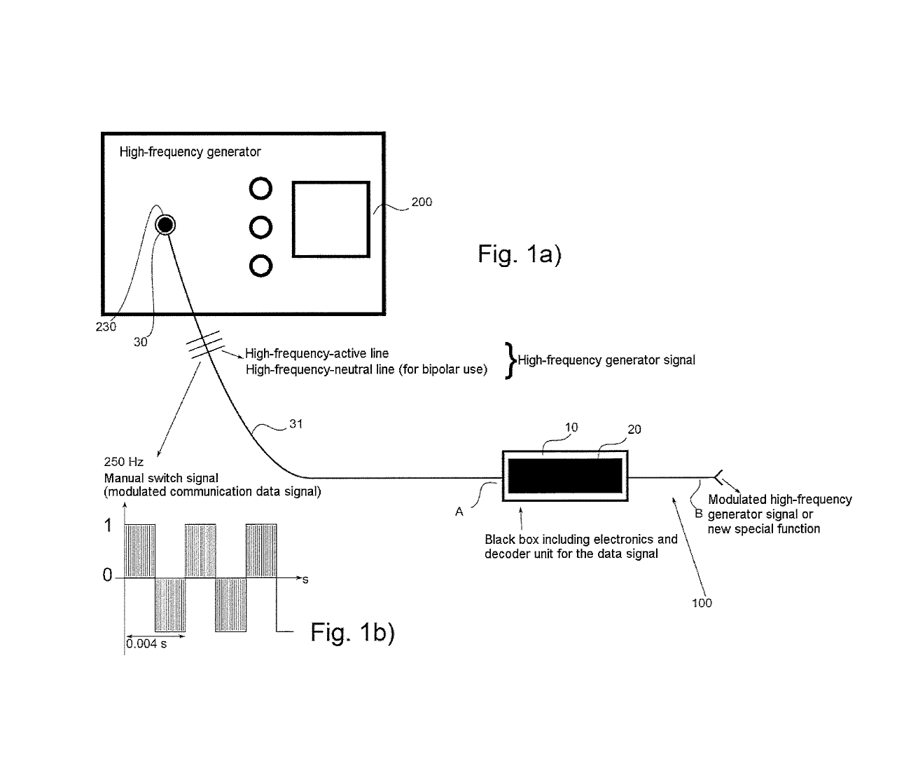

FIG. 1a) depicts a schematic representation of an exemplary specific embodiment of an electrosurgical instrument according to the present invention which is connected to a high-frequency generator;

FIG. 1b) depicts a modulated hand switch signal;

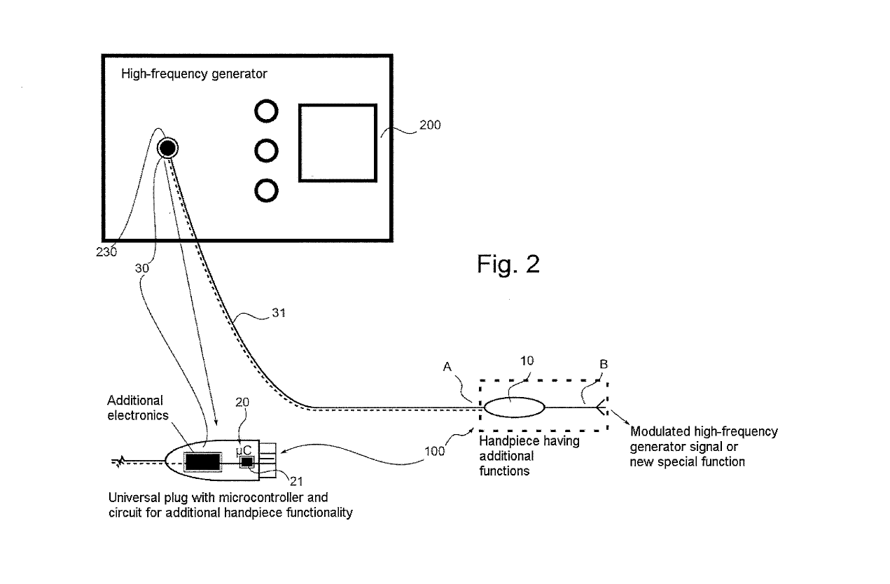

FIG. 2 depicts a schematic representation of an additional exemplary specific embodiment of an electrosurgical instrument according to the present invention and of a high-frequency generator.

A high-frequency generator 200 in figure 1a) comprises a device connector 230 for connecting an electrosurgical instrument 100, wherein high-frequency energy is able to be transferred from the high-frequency generator 200 to the electrosurgical instrument 100 via the device connector 230.

The electrosurgical instrument 100 comprises a handpiece 10 which has a connection side A and a treatment side B. For example, electrosurgical treatment electrodes (not shown) may be situated on the treatment side B of the handpiece 10. The electrosurgical instrument 100 furthermore comprises a connecting plug 30 which is presently electrically connected to the high-frequency generator 200, wherein the connecting plug 30 and the handpiece 10 in turn are electrically in contact via a connecting line 31. The electrosurgical instrument 100 comprises a control unit 20 which is adapted to actively control the voltage waveform of high-frequency energy which is output on the treatment side B of the handpiece 10. As is apparent from FIG. 1, the control unit 20 is situated in the handpiece 10 itself.

The instrument 100 comprises a feedback channel to the high-frequency generator 200, and the control unit 20 is adapted to control a voltage waveform of the high-frequency energy which is specified by the high-frequency generator 200 to the instrument 100, via the feedback channel. The feedback channel is presently provided via a modulated hand switch signal. The hand switch signal is, like the high-frequency energy itself, transmitted via the connecting line 31. FIG. 1b) depicts such a modulated (frequency-modulated) hand switch signal having a modulation frequency of 250 Hz.

Simultaneously, the control unit 20 is synchronized with a change in the voltage waveform of the high-frequency energy provided by the high-frequency generator 200 via a via the modulated switch signal. For this purpose, the control unit 20 comprises a decoder unit (not shown) in order to demodulate the modulated hand switch signal.

Another exemplary specific embodiment is depicted in FIG. 2. The high-frequency generator 200, which corresponds to the high-frequency generator of FIG. 1, comprises a device connector 230 for outputting high-frequency energy to the electrosurgical instrument 100. Unlike the instrument described with reference to FIG. 1, in the electrosurgical instrument 100 of FIG. 2, the control unit 20 is situated in the connecting plug 30 of the electrosurgical instrument 100. The control unit 20 comprises a microcontroller 21 which is adapted to output an instrument identification of the electrosurgical instrument 100 to the high-frequency generator 200. Furthermore, the microcontroller 21 is adapted to emulate an instrument identification of a passive non-volatile memory with respect to the high-frequency generator 200.

Also in the present specific embodiment, the control unit 20 assumes the enhanced functionality of the instrument 100 using its microcontroller 21, i.e., for example, the output of a voltage waveform on the treatment side B of the handpiece 10, which the high-frequency generator 200 is not able to provide directly.

During an initialization of the instrument 100, which is carried out when the instrument 100 is connected to the high-frequency generator 200, the microcontroller 21 contained by the control unit 20 initially emulates the protocol of a passive non-volatile memory used in a conventional electrosurgical instrument (not shown). Subsequently, the high-frequency generator 200, which has been provided with a software update, establishes a communication connection with the instrument 100 via the feedback channel implemented via the connecting line 31, thereby making it possible to achieve enhanced functionality of the instrument 100 via its control unit 20.

1. Electrosurgical instrument including a handpiece which has a connection side (A) and a treatment side (B), and including a connecting plug which is electrically connected to the connection side (A) of the handpiece and is electrically connectable to a high-frequency generator, wherein the electrosurgical instrument includes a control unit which is designed to actively control the voltage waveform of high-frequency energy which is output on the treatment side (B) of the handpiece.

2. Instrument according to claim 1, wherein the control unit is designed to modify, in particular to modulate, high-frequency energy supplied by the high-frequency generator as required, and to output this modified, in particular modulated, high-frequency energy on the treatment side (B) of the handpiece.

3. Instrument according to claim 1, wherein the instrument has a feedback channel to the high-frequency generator and the control unit is designed to control a voltage waveform of the high-frequency energy which is output by the high-frequency generator to the instrument via the feedback channel.

4. Instrument according to claim 1, wherein the control unit is designed to switch between different high-frequency voltage modes, in particular, between a cutting mode and a coagulation mode, which are output by the high-frequency generator to the instrument.

5. Instrument according to claim 1, wherein the feedback channel is provided via a modulated manual switch signal.

6. Instrument according to claim 1, wherein the control unit is synchronized with a change in the voltage waveform of the high-frequency energy which is output by the high-frequency generator via a modulated switch signal.

7. Instrument according to claim 1, wherein the control unit is controllable via a modulated manual switch signal which is output by the high-frequency generator.

8. Instrument according to claim 1, wherein the control unit is supplied with power via a manual switch line.

9. Instrument according to claim 1, wherein the control unit includes a microcontroller which is designed to output an instrument identification of the electrosurgical instrument to a high-frequency generator, and/or the microcontroller is designed to emulate an instrument identification of a passive nonvolatile memory with respect to a high-frequency generator.

10. High-frequency surgical device including an electrosurgical instrument according to claim 1, and including a high-frequency generator.
